(19) United States
(12) Patent Application Publication
(10) Pub. No.: US 2022/0068497 A1
(43) Pub. Date: Mar. 3, 2022

(54) MEDICAL INFORMATION PROCESSING APPARATUS, MEDICAL INFORMATION PROCESSING SYSTEM, AND MEDICAL INFORMATION PROCESSING METHOD

(71) Applicant: CANON MEDICAL SYSTEMS CORPORATION, Otawara-shi (JP)

(72) Inventors: Kohei SHINOHARA, Nasushiobara (JP); Maki MINAKUCHI, Utsunomiya (JP); Shuhei BANNAE, Utsunomiya (JP); Hisaaki OOSAKO, Utsunomiya (JP)

(73) Assignee: CANON MEDICAL SYSTEMS CORPORATION, Otawara-shi (JP)

(21) Appl. No.: 17/465,055

(22) Filed: Sep. 2, 2021

(30) Foreign Application Priority Data
Sep. 2, 2020 (JP) 2020-147538

(51) Int. Cl. G16H 50/70; G16H 50/20; G16H 10/60; G16H 30/40

(52) U.S. Cl. CPC G16H 50/70; G16H 30/40; G16H 10/60; G16H 50/20

(57) ABSTRACT

A medical information processing apparatus according to an embodiment includes processing circuitry configured to obtain a plurality of illness candidates; collect information serving as the evidence for determining the illness of the patient from among the plurality of illness candidates, and obtain a score for each illness candidate based on that information; identify, based on the scores, an examination candidate meant for supporting the diagnosis of the patient; and perform output based on the examination candidate.

### CROSS-REFERENCE TO RELATED APPLICATIONS

This application is based upon and claims the benefit of priority from Japanese Patent Application No. 2020-147538, filed on Sep. 2, 2020; the entire contents of which are incorporated herein by reference.

### FIELD

Embodiments described herein relate generally to a medical information processing apparatus, a medical information processing system, and a medical information processing method.

### BACKGROUND

From a patient visiting a hospital, a variety of medical information is collected via a medical interview and examination, and that information is used in performing diagnosis. Apart from the medical information collected for use in diagnosis, there is a variety of other information that serves as the evidence in regard to performing diagnosis. However, such information is enormous in volume, and utilization thereof is not an easy task.

### BRIEF DESCRIPTION OF THE DRAWINGS

FIG. 1 is a block diagram illustrating an exemplary configuration of a medical information processing system according to a first embodiment;

FIG. 2A is a diagram for explaining the operations performed by a processing circuit according to the first embodiment;

FIG. 2B is a diagram for explaining the operations performed by the processing circuit according to the first embodiment;

FIG. 2C is a diagram illustrating the operations performed when there is a deficit of the information serving as the evidence according to the first embodiment;

FIG. 3 is a diagram for explaining about examination candidates according to the first embodiment;

FIG. 4A is a diagram for explaining about an examination candidate according to the first embodiment;

FIG. 4B is a diagram for explaining about the examination candidates according to the first embodiment;

FIG. 4C is a diagram for explaining the examination candidates according to the first embodiment;

FIG. 5 is a flowchart for explaining a sequence of operations performed in a medical information processing apparatus according to the first embodiment;

FIG. 6 is a diagram illustrating an example of a feedback according to a second embodiment;

FIG. 7 is a diagram illustrating an example of a feedback according to a third embodiment;

FIG. 8 is a diagram illustrating an example of a feedback according to the third embodiment; and

FIG. 9 is a block diagram illustrating an exemplary configuration of the medical information processing apparatus according to the third embodiment.

### DETAILED DESCRIPTION

A medical information processing apparatus comprises processing circuitry. The processing circuitry is configured to obtain a plurality of illness candidates, collect information serving as evidence for determining illness of a patient from among the plurality of illness candidates, and obtain a score for each of the plurality of illness candidates based on the information, identify, based on the scores, an examination candidate meant for supporting diagnosis of the patient, and perform output based on the examination candidate.

Exemplary embodiments of a medical information processing apparatus, a medical information processing system, and a medical information processing method are described below in detail with reference to the accompanying drawings.

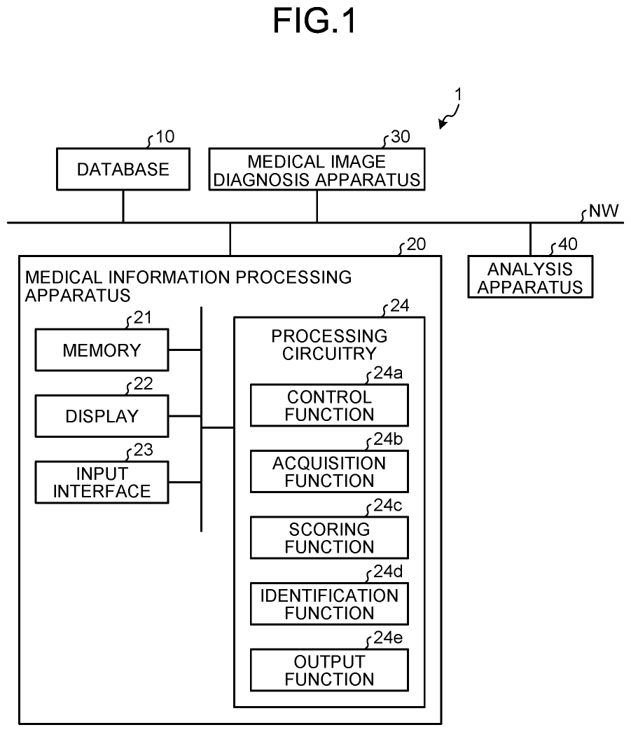

In the embodiments, the explanation is given for a medical information processing system 1 that includes a medical information processing apparatus 20. For example, as illustrated in FIG. 1, the medical information processing system 1 includes a database 10, the medical information processing apparatus 20, a medical image diagnosis apparatus 30, and an analysis apparatus 40. FIG. 1 is a block diagram illustrating an exemplary configuration of the medical information processing system 1 according to a first embodiment. The database 10, the medical information processing apparatus 20, the medical image diagnosis apparatus 30, and the analysis apparatus 40 are connected to each other via a network NW.

As long as a connection with the network NW can be established, the devices included in the medical information processing system 1 can be installed at arbitrary installation locations. For example, the database 10, the medical information processing apparatus 20, the medical image diagnosis apparatus 30, and the analysis apparatus 40 can be installed in mutually different facilities. Thus, the network NW can be configured as a closed local network among the facilities, or can be a network configured via the Internet.

The database 10 represents a data storage device used to store a variety of information. For example, as far as the database 10 is concerned, an arbitrary memory device is installed either internally or externally, and a variety of information obtained via the network NW is managed in the form of a database in the memory device. Alternatively, the database 10 can be implemented using a group of servers (a cloud) that is connected to the medical information processing system 1 via the network NW. Regarding the information stored in the database 10, the explanation is given later.

The medical image diagnosis apparatus 30 collects medical images from a patient P. Examples of the medical image diagnosis apparatus 30 include an X-ray diagnosis apparatus, an X-ray CT apparatus (CT stands for Computed Tomography), an MRI apparatus (MRI stands for Magnetic Resonance Imaging), an ultrasonic diagnosis apparatus, a SPECT apparatus (SPECT stands for Single Photon Emission Computed Tomography), and a PET apparatus (PET stands for Positron Emission computed Tomography). The medical images collected by the medical image diagnosis apparatus 30 represent a part of the information serving as the evidence in regard to performing diagnosis of the patient P. Meanwhile, the medical information processing system 1 can include a plurality of medical image diagnosis apparatus 30.

The analysis apparatus 40 performs analysis related to the patient P. For example, the analysis apparatus 40 analyzes the specimen material such as the blood collected from the patient P, and analyzes the medical images collected by the medical image diagnosis apparatus 30 from the patient P. As an example, the analysis apparatus 40 performs computer-aided diagnosis (CAD) with respect to the medical images collected from the patient P and, in case a lesion is suspected, outputs the analysis result by putting a mark at the concerned position in the medical images. Herein, the analysis result obtained by the analysis apparatus 40 represents a part of the information serving as the evidence in regard to performing diagnosis the patient P. Meanwhile, the analysis apparatus 40 represents an example of an analyzing unit. Moreover, the medical information processing system 1 can include a plurality of analysis apparatus 40.

From the database 10, the medical image diagnosis apparatus 30, and the analysis apparatus 40; the medical information processing apparatus 20 collects the information serving as the evidence in regard to performing diagnosis of the patient P; and performs various operations as explained below. The medical information processing apparatus 20 includes, for example, a memory 21, a display 22, an input interface 23, and processing circuitry 24 as illustrated in FIG. 1.

The memory 21 is implemented using, for example, a semiconductor memory device such as a random access memory (RAM) or a flash memory; or a hard disk; or an optical disk. For example, the memory 21 is used to store the information collected from the database 10, the medical image diagnosis apparatus 30, and the analysis apparatus 40. Moreover, the memory 21 is used to store computer programs that are meant to enable the circuits included in the medical information processing apparatus 20 to implement their respective functions. Meanwhile, the memory 21 can alternatively be implemented using a group of servers (a cloud) that is connected to the medical information processing apparatus 20 via the network NW.

The display 22 is used to display a variety of information. For example, the display 22 displays a graphical user interface (GUI) for receiving various instructions and settings from the user via the input interface 23. Moreover, the display 22 displays examination candidates (explained later). Examples of the display 22 include a liquid crystal display and a cathode ray tube (CRT) display. The display 22 either can be a desktop-type display, or can be configured using a tablet terminal capable of performing wireless communication with the main body of the medical information processing apparatus 20.

In the explanation given with reference to FIG. 1, the medical information processing apparatus 20 includes the display 22. Alternatively, the medical information processing apparatus 20 can include a projector in place of or in addition to the display 22. Under the control of the processing circuitry 24, the projector can perform projection on a screen, a wall, the floor, or the body surface of the patient P. As an example, the projector can perform projection on an arbitrary plane, an arbitrary object, or an arbitrary space according to projection mapping.

The input interface 23 receives various types of input operations from the user; converts the received input operations into electrical signals; and outputs the electrical signals to the processing circuitry 24. For example, the input interface 23 is implemented using a mouse or a keyboard; a trackball; switches; buttons; a joystick; a touchpad for performing input operations by touching its operation screen; a touchscreen in which a display screen and a touchpad are integrated; a contactless input circuit in which an optical sensor is used; or a voice input circuit. Alternatively, the input interface 23 can be configured using a tablet terminal capable of performing wireless communication with the main body of the medical information processing apparatus 20. Still alternatively, the input interface 23 can be a circuit that receives input operations from the user based on motion capturing. As an example, the input interface 23 can process signals that are obtained via a tracker or can process user-related images that are collected, and can receive the body motion or the line of sight of the user as an input operation. Meanwhile, the input interface 23 is not limited to include a physical operation component such as a mouse or a keyboard.

Alternatively, as an example of the input interface 23, it is also possible to consider an electrical signal processing circuit that receives electrical signals corresponding to input operations from an external input device installed separately from the medical information processing apparatus 20, and that outputs electrical signals to the processing circuitry 24.

The processing circuitry 24 executes a control function 24a, an acquisition function 24b, a scoring function 24c, an identification function 24d, and an output function 24e; and thus controls the operations of the entire medical information processing apparatus 20. The acquisition function 24b represents an example of an obtaining unit. The scoring function 24c represents a scoring unit. The identification function 24d represents an example of an identifying unit. The output function 24e represents an example of an output unit.

For example, the processing circuitry 24 reads a computer program, which corresponds to the control function 24a, from the memory 21 and executes it; and resultantly controls various functions such as the acquisition function 24b, the scoring function 24c, the identification function 24d, and the output function 24e based on various types of input operations received from the user via the input interface 23.

Moreover, the processing circuitry 24 reads a computer program, which corresponds to the acquisition function 24b, from the memory 21 and executes it; and resultantly obtains a plurality of illness candidates. Furthermore, the processing circuitry 24 reads a computer program, which corresponds to the scoring function 24c, from the memory 21 and executes it; and resultantly collects information serving as the evidence for distinguishing the illness of the patient P from among the illness candidates, and obtains scores for the illness candidates. Moreover, the processing circuitry 24 reads a computer program, which corresponds to the identification function 24d, from the memory 21 and executes it; and identifies, based on the scores, examination candidates meant for supporting the diagnosis of the patient P. Furthermore, the processing circuitry 24 reads a computer program, which corresponds to the output function 24e, from the memory 21 and executes it; and resultantly performs output based on the examination candidates. Regarding the functions of the processing circuitry 24, the detailed explanation is given later.

In the medical information processing apparatus 20 illustrated in FIG. 1, the processing functions are stored as computer-executable programs in the memory 21. The processing circuitry 24 is a processor that reads the computer programs from the memory 21 and executes them, so that the functions corresponding to the computer programs are implemented. In other words, after having read the computer programs, the processing circuitry 24 gets equipped with the functions corresponding to the read computer programs.

Meanwhile, with reference to FIG. 1, the control function 24a, the acquisition function 24b, the scoring function 24c, the identification function 24d, and the output function 24e are implemented in a single processing circuitry 24. Alternatively, the processing circuitry 24 can be configured by combining a plurality of independent processors, and each processor can execute computer programs and implement functions. Still alternatively, the processing functions of the processing circuitry 24 can be implemented by appropriately dispersing or integrating them in a single processing circuit or a plurality of processing circuits.

Still alternatively, the processing circuitry 24 can implement the functions using the processor of an external device that is connected via the network NW. For example, in addition to reading computer programs corresponding to the functions from the memory 21 and executing them, the processing circuitry 24 also uses a group of servers (a cloud), which is connected to the medical information processing apparatus 20 via the network NW, as the calculation resources; and thus implements the functions illustrated in FIG. 1.

Till now, the explanation was given about an exemplary configuration of the medical information processing system 1 that includes the medical information processing apparatus 20. With such a configuration, the processing circuitry 24 of the medical information processing apparatus 20 performs operations as explained below and effectively utilizes the information that serves as the evidence in regard to diagnosing the patient P.

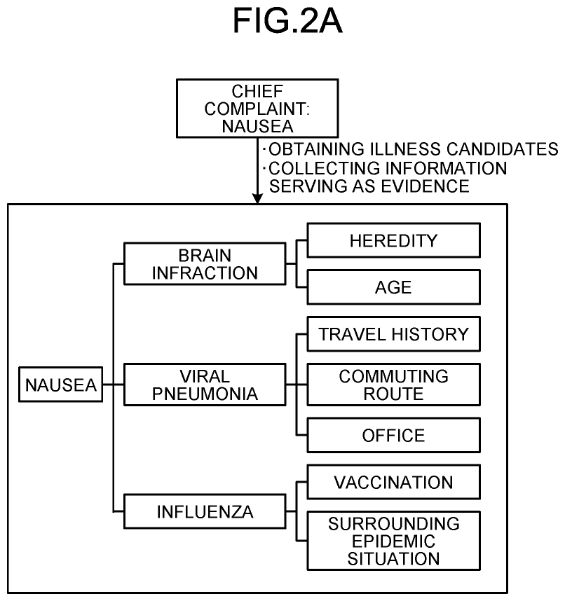

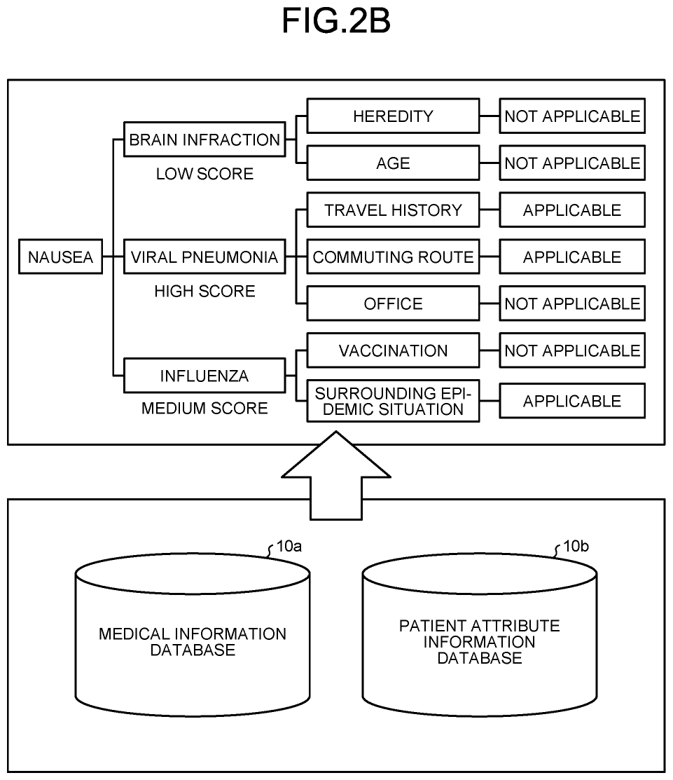

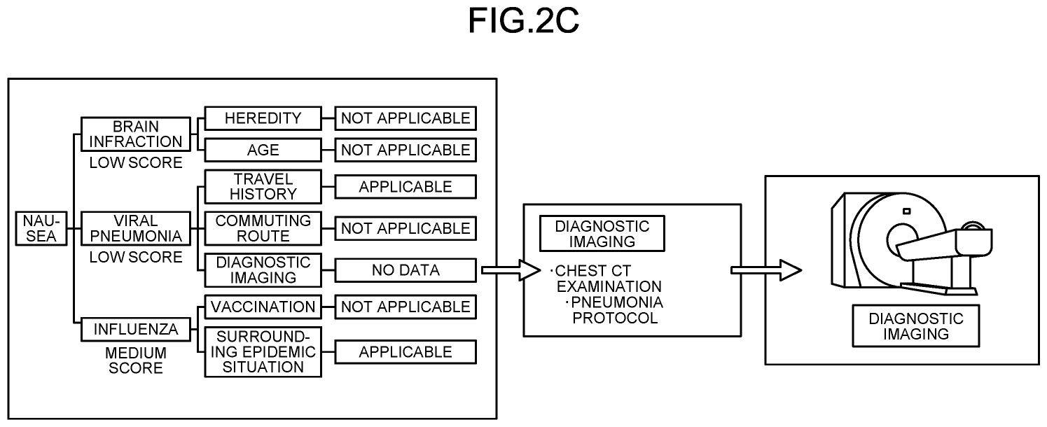

Firstly, after visiting a hospital or a clinic, the patient P describes the symptoms at the reception or during the medical interview. For example, as illustrated in FIG. 2A, the patient P describes “nausea” as the chief complaint. FIG. 2A is a diagram for explaining the operations performed by the processing circuitry 24 according to the first embodiment.

The acquisition function 24b obtains the chief complaint of the patient P. For example, the chief complaint of the patient P is registered in a system such as a hospital information system (HIS) or a radiology information system (RIS); and the acquisition function 24b can automatically obtain the chief complaint from the system. Alternatively, the acquisition function 24b can obtain the chief complaint of the patient P by receiving input from the user via the input interface 23.

Then, the acquisition function 24b obtains a plurality of illness candidates based on the chief complaint of the patient P. That is, based on the chief complaint of “nausea” described by the patient P, the acquisition function 24b obtains a plurality of illness candidates in which “nausea” is included as a symptom.

For example, the acquisition function 24b obtains, in advance, association information in which symptoms and illnesses are associated; and obtains the illnesses associated to the chief complaint of “nausea” as the illness candidates for the patient P. The association information either can be created by the acquisition function 24b, or can be manually created by the user, or can be created in an external device other than the medical information processing apparatus 20. As an example, based on the clinical record created in the past, the acquisition function 24b can obtain the definite diagnosis about the symptoms described by the patient and the illness name; and can accordingly generate the association information. The association information is stored in, for example, the memory 21; and the acquisition function 24b can read the association information from the memory 21 and use it.

As another example, the acquisition function 24b implements a predetermined algorithm and obtains a plurality of illness candidates. The algorithm can be implemented using, for example, a machine learning method. For example, based on the clinical record created in the past, the acquisition function 24b obtains the definite diagnosis about the symptoms described by the patient and the illness name. Then, the acquisition function 24b performs machine learning in which the symptoms are treated as input-side data and the definite diagnosis of the illness name is treated as output-side data, and generates an already-learnt model functionalized to receive input of the symptoms and to output the illness candidates. The already-learnt model can be configured using, for example, a neural network. Moreover, the already-learnt model can be generated in an external device other than the medical information processing apparatus 20. The already-learnt model is stored in, for example, the memory 21; and the acquisition function 24b can read the already-learnt model from the memory 21 and use it.

Meanwhile, with reference to FIG. 2A, although a plurality of illness candidates is obtained based on the symptoms such as the chief complaint of “nausea”, the method of obtaining a plurality of illness candidates is not limited to that method. Alternatively, for example, a plurality of illness candidates can be obtained based on the result of examination of the patient P. Still alternatively, for example, the user sets a plurality of illness candidates, and the acquisition function 24b obtains a plurality of illness candidates by receiving an input operation from the user.

Subsequently, the scoring function 24c collects the information that serves as the evidence in regard to diagnosing the patient P. More particularly, the information serving as the evidence is the information that enables determination of the illness of the patient P from among the illness candidates obtained by the acquisition function 24b. In other words, the information serving as the evidence represents the information serving as the criteria for determining the illness or represents the reference information for determining the illness.

With reference to FIG. 2A, the acquisition function 24b obtains “brain infraction”, “viral pneumonia” and “influenza” as the illness candidates. Moreover, with reference to FIG. 2A, the scoring function 24c collects “heredity”, “age”, “travel history”, “commuting route”, “office”, “vaccination”, and “surrounding epidemic situation” as the information serving as the evidence. More particularly, the scoring function 24c collects “heredity” and “age” as the information serving as the evidence for “brain infraction”. That is, “heredity” and “age” represent the factors affecting the incidence rate of “brain infraction”, and serve as the criteria for determining whether the patient P is suffering from “brain infraction”. In an identical manner, the scoring function 24c collects “travel history”, “commuting route”, and “office” as the information serving as the evidence for “viral pneumonia”. Moreover, the scoring function 24c collects “vaccination” and “surrounding epidemic situation” as the information serving as the evidence for “influenza”.

For example, as illustrated in FIG. 2B, from a medical information database 10a and a patient attribute information database 10b, the scoring function 24c can collect the information serving as the evidence. FIG. 2B is a diagram for explaining the operations performed by the processing circuitry 24 according to the first embodiment. The medical information database 10a and the patient attribute information database 10b represent examples of the database 10.

The medical information database 10a is used to store the medical information about a plurality of patients including the patient P. For example, the medical information database 10a is a server of an HIS, an RIS, or a PACS (which stands for Picture Archiving and Communication System).

The medical information contains a variety of information collected from the patient with the purpose of performing diagnosis. As an example, the medical information contains the medical images collected from the patient in the past, and contains the result of the analysis operations performed for the patient in the past. Moreover, the medical information also contains the basic information of the patient, the blood relationships, and the surrounding information. The basic information represents information such as the address and the birthdate of the patient. The blood relationships represent information such as the names of predetermined relatives such as the parents of the patient, and the patient ID. The surrounding information indicates the epidemic situation of various illnesses around the house of the patient and at the workplace of the patient. The basic information, the blood relationships, and the surrounding information is obtained, for example, at the reception or during the medical interview of the patient at the time of a visit to the hospital; and is registered in the medical information database 10a.

The patient attribute information database 10b is not limited to be used for managing the information collected for the diagnostic purpose, but is also used to manage patient attribute information collected under a variety of circumstances. The patient attribute information database 10b can be a database administered by a specific hospital or a specific business enterprise, or can be a publicly-administered database.

Examples of the patient attribute information include the following information of the patient: national identification number, travel history, location information, action information, school, office, work information, and residential history. Thus, the patient attribute information database 10b is, for example, a database for centrally managing the patient attribute information with the focus on each patient.

Meanwhile, the patient attribute information database 10b can be an assembly of a plurality of databases. In that case too, the patient attribute information in each database can be linked using the national identification number, so that the databases can be centrally managed.

For example, based on the blood relationship information stored in the medical information database 10a and based on the national identification number stored in the patient attribute information database 10b, the scoring function 24c collects the information indicating “heredity: not applicable” as illustrated in FIG. 2B. Moreover, for example, based on the basic information stored in the medical information database 10a and based on the national identification number stored in the patient attribute information database 10b, the scoring function 24c collects the information indicating “age: not applicable”. Furthermore, for example, based on the travel history, the location information, the action information, and the residential history as stored in the patient attribute information database 10b, the scoring function 24c collects the information indicating “travel history: applicable”. Moreover, for example, based on the basic information stored in the medical information database 10a and based on the office and the work information stored in the patient attribute information database 10b, the scoring function 24c collects the information indicating “commuting route: applicable”. Furthermore, for example, based on the basic information and the surrounding information stored in the medical information database 10a and based on the office and the work information stored in the patient attribute information database 10b, the scoring function 24c collects the information indicating “office: not applicable”. Moreover, for example, based on the action information stored in the patient attribute information database 10b, the scoring function 24c collects the information indicating “vaccination: not applicable”. Furthermore, for example, based on the surrounding information stored in the medical information database 10a and based on the school, the office, and the work information stored in the patient attribute information database 10b, the scoring function 24c collects the information indicating “surrounding epidemic situation: applicable”.

Moreover, the scoring function 24c can collect the information serving as the evidence also from the devices other than the medical information database 10a and the patient attribute information database 10b. For example, the scoring function 24c can collect, as the information serving as the evidence, the medical images of the patient P as collected by the medical image diagnosis apparatus 30 and the analysis operation performed for the patient P by the analysis apparatus 40. Moreover, the scoring function 24c can also receive input of the information, which serves as the evidence, via the input interface 23.

Then, based on the information serving as the evidence, the scoring function 24c obtains the scores of the illness candidates. That is, the scoring function 24c assigns scores to the illness candidates. Meanwhile, there is no particular restriction on the method of obtaining the scores. For example, the scoring function 24c can calculate the scores using a predetermined equation in which the information serving as the evidence represents the variables; or can read the scores from a predetermined table in which the information serving as the evidence is associated to scores.

Meanwhile, the scores can be in the form of numerical values or can be in the form of data other than numerical values. The scores indicate the evaluation of the illness candidates, and there is no particular restriction on the specific form of the scores. For example, the scores can be in the form of ranks such as “low score”, “medium score”, and “high score” as illustrated in FIG. 2B.

For example, in the case illustrated in FIG. 2B, based on the information indicating “heredity: not applicable” and “age: not applicable”, the scoring function 24c obtains the score about the illness candidate “brain infraction”. Moreover, based on the information indicating “travel history: applicable”, “commuting route: applicable”, and “office: not applicable”, the scoring function 24c obtains the score about the illness candidate “viral pneumonia”. Furthermore, based on the information indicating “vaccination: not applicable” and “surrounding epidemic situation: applicable”, the scoring function 24c obtains the score about the illness candidate “influenza”.

For example, in the case illustrated in FIG. 2B, “heredity” as well as “age” is not applicable as far as the illness candidate “brain infraction” is concerned. Hence, the scoring function 24c obtains “0” as the score. Moreover, from among “travel history”, “commuting route”, and “office”, two items are applicable as far as the illness candidate “viral pneumonia” is concerned. Hence, the scoring function 24c obtains “⅔” as the score. Furthermore, one of “vaccination” and “surrounding epidemic situation” is applicable as far as the illness candidate “influenza” is concerned. Hence, the scoring function 24c obtains “½” as the score.

Meanwhile, the scoring function 24c can obtain the scores also by assigning weights to the information serving as the evidence. For example, the scoring function 24c assigns the weight of “3:1” with respect to “vaccination” and “surrounding epidemic situation”. In that case, with reference to FIG. 2B, since only “surrounding epidemic situation” is applicable as far as the illness candidate “influenza” is concerned, the scoring function 24c obtains “¼” as the score.

In FIG. 2B is illustrated the case in which there is no deficit of the information serving as the evidence, and the score can be obtained for each illness candidate. However, it is possible to think of a case in which there is a deficit of the information required to obtained the scores.

For example, in the case illustrated in FIG. 2B, there can be a case in which it is not clear whether the patient P has taken vaccination for the current year. In that case, the output function 24e notifies the user about the facts that there is a deficit of the information serving as the evidence and that the score for the illness candidate “influenza” cannot be obtained. In that regard, when the user takes the medical interview of the patient P, the scoring function 24c can obtain the information about “vaccination: not applicable” and accordingly obtain the score about the illness candidate “influenza”.

As another example, as illustrated in FIG. 2C, there can be a case in which diagnostic imaging is considered necessary for obtaining the score for the illness candidate “viral pneumonia”, but the image data is not available. In that case, the identification function 24d identifies the examination candidate for obtaining the deficit information. FIG. 2C is a diagram illustrating the operations performed when there is a deficit of the information serving as the evidence according to the first embodiment.

For example, as illustrated in FIG. 2C, the identification function 24d identifies chest CT examination conforming to the pneumonia protocol as the examination candidate. Moreover, the output function 24e notifies the user about the facts that the score about the illness candidate “viral pneumonia” cannot be obtained due to the deficit of the information serving as the evidence, and that the chest CT examination is required to obtain the deficit information. If the user determines that chest CT examination is required, then chest CT examination of the patient P is performed based on the pneumonia protocol. Furthermore, the scoring function 24c uses the result of the chest CT examination as the information serving as the evidence, and obtains the score about the illness candidate “viral pneumonia”.

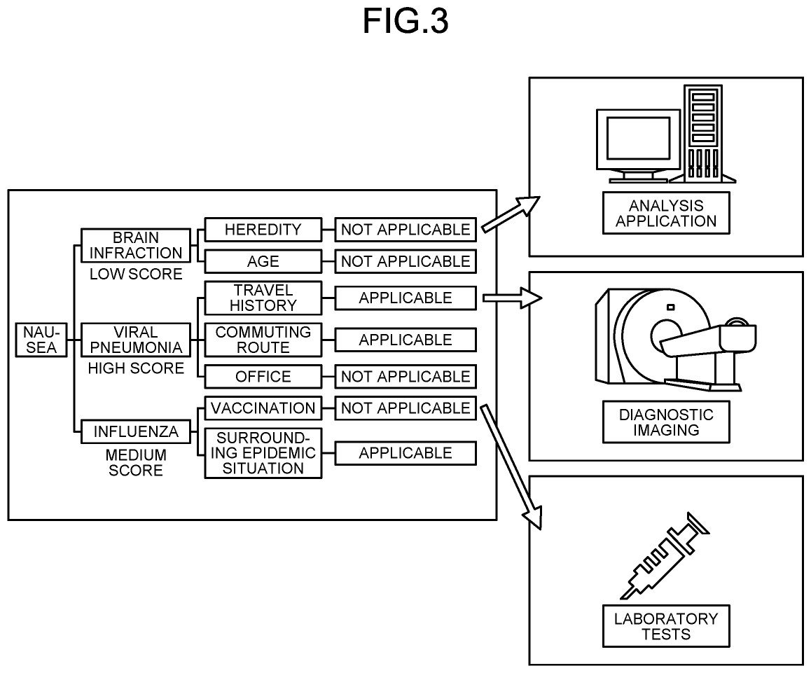

After the score is obtained for each of a plurality of illness candidates, the identification function 24d identifies, based on the scores, the examination candidates meant for supporting the diagnosis of the patient P. For example, as illustrated in FIG. 3, the identification function 24d identifies the examination candidates from among various types of examinations such as analysis based on an analysis application; diagnostic imaging; and laboratory tests. FIG. 3 is a diagram for explaining about the examination candidates according to the first embodiment.

For example, in the case illustrated in FIG. 3, the illness candidate “viral pneumonia” has a high score, thereby indicating the possibility that the patient P is suffering from “viral pneumonia”. If the illness candidate “viral pneumonia” is of the critical type, then the identification function 24d identifies detailed examination for “viral pneumonia” as the examination candidate. An illness candidate of the critical type is, for example, an illness having a high fatality rate, having a high risk of leaving after-effects, and having a high infectability. The detailed examination represents, for example, the examination enabling definite diagnosis of the illness and enabling determination of the severity.

Subsequently, the output function 24e outputs the examination candidate identified by the identification function 24d. For example, the output function 24e notifies the user, such as the primary doctor who is diagnosing the patient P, about the examination candidate identified by the identification function 24d. For example, in the case illustrated in FIG. 3, the output function 24e displays, in the display 22, the following: the fact that the patient P has a high score for “viral pneumonia”; the fact that the illness “viral pneumonia” is a critical illness and it is recommended to perform detailed examination; and the examination candidate recommended as the detailed examination of “viral pneumonia”.

Upon receiving the notification from the output function 24e, the user studies the notified examination candidate and, if the examination is determined to be necessary, can ensure that examination based on the examination candidate is performed. For example, based on the notified examination candidate, the user issues an analysis order to the analysis apparatus 40 and ensures that analysis is performed with the use of an analysis application. Moreover, for example, based on the notified examination candidate, the user makes a diagnostic imaging order to the medical image diagnosis apparatus 30 and ensures that images of the patient P are collected.

Meanwhile, instead of notifying the user about the examination candidate identified by the identification function 24d, the output function 24e itself can make an order for examination. For example, based on the examination candidate identified by the identification function 24d, the output function 24e issues an analysis order to the analysis apparatus 40 so that analysis is performed with the use of an analysis application. Moreover, for example, based on the examination candidate identified by the identification function 24d, the output function 24e makes a diagnostic imaging order to the medical image diagnosis apparatus 30 so that images of the patient P are collected. Meanwhile, in order to provide rationalization for issuing such orders, the output function 24e can attach the scores, which are obtained by the scoring function 24c, to the orders. Then, the output function 24e notifies the user about the result of ordered examinations.

In the case illustrated in FIG. 3, the medical information processing apparatus 20 collects a variety of information that serves as the evidence for determining the illness of the patient P from among a plurality of illness candidates; and, if there is a possibility that the patient is suffering from a critical illness, enables performing the detailed examination. Thus, the medical information processing apparatus 20 can effectively utilize the information serving as the evidence in regard to performing diagnosis, and thus support the user to perform diagnosis.

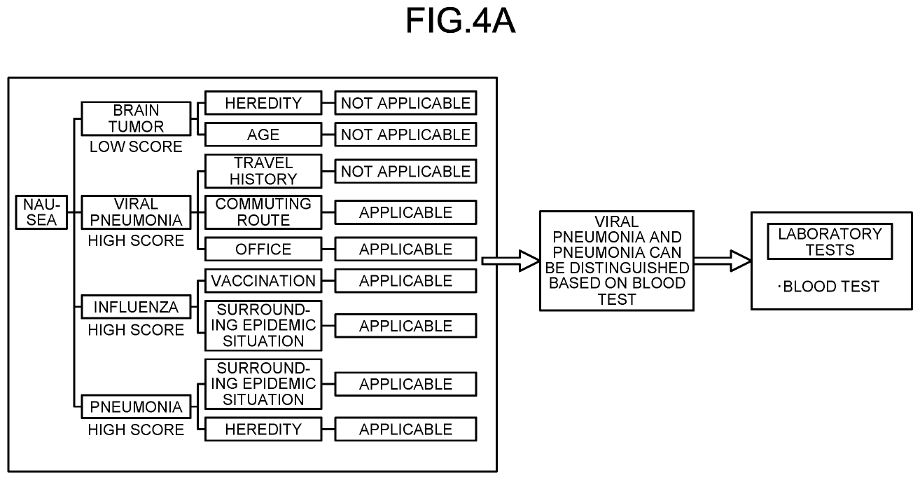

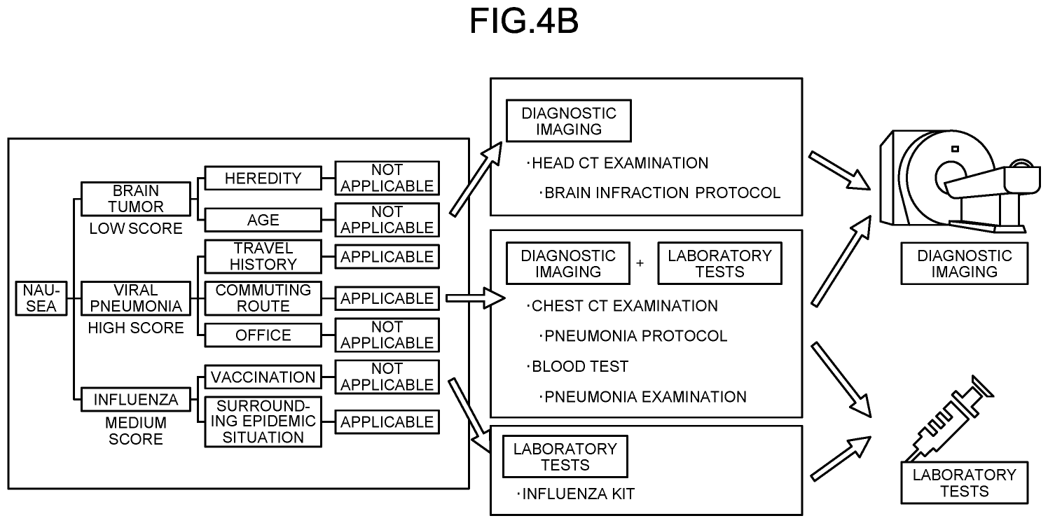

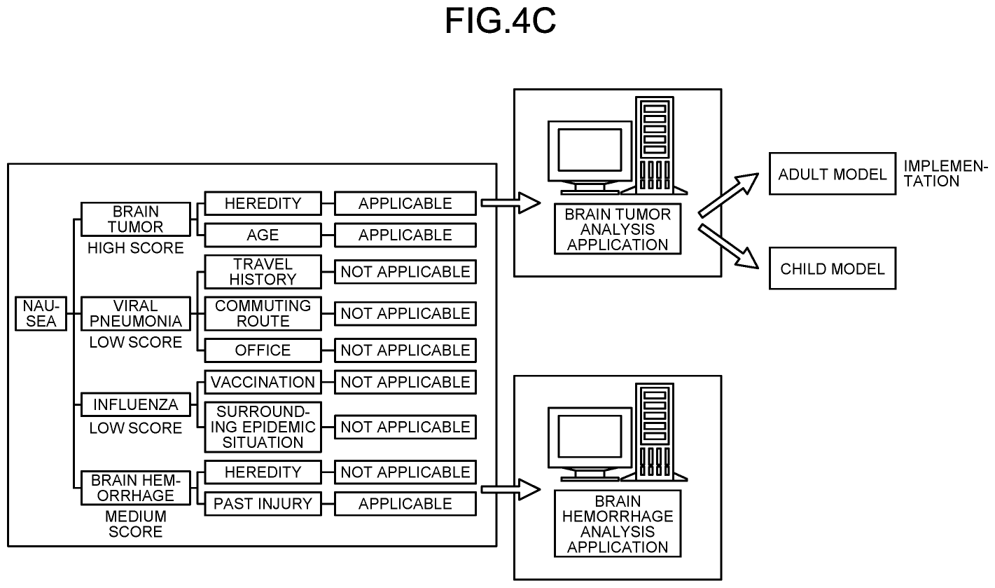

Given below is the explanation of another example about the illness candidates identified by the identification function 24d. For example, in FIG. 3 is illustrated the case in which there is a single illness candidate having a high score. Alternatively, for example, it is also possible to think of a case in which a plurality of illness candidates has a high score as illustrated in FIG. 4A. In that case, the identification function 24d identifies, as the examination candidate, the examination for determining the illness of the patient P from among the illness candidates having a high score. FIG. 4A is a diagram for explaining about the examination candidate according to the first embodiment.

More particularly, in the case illustrated in FIG. 4A, from among four illness candidates, the illness candidates “viral pneumonia”, “influenza”, and “pneumonia” have a high score, thereby indicating that the patient P might be suffering from those illnesses. In the following explanation, the illness candidates that are indicated to be the likely illnesses of the patient P on account of their scores are referred to as specified illness candidates. The identification function 24d identifies, as the examination candidate, the examination for determining the illness of the patient P from among the specified illness candidates, namely, “viral pneumonia”, “influenza”, and “pneumonia”. For example, since “viral pneumonia” and “pneumonia” can be distinguished based on a blood test, the identification function 24d identifies a blood test as the examination candidate.

The output function 24e either notifies the user about the fact that a blood test is identified as the examination candidate, or issues a blood test order. Then, depending on the result of the blood test, it becomes possible to determine whether the patient P is suffering from “viral pneumonia” or “pneumonia”. On the other hand, according to the blood test, if it becomes clear that the patient P is neither suffering from “viral pneumonia” nor suffering from “pneumonia”, then it can be inferred that “influenza” is the illness.

As illustrated in FIG. 4A, the medical information processing apparatus 20 collects a variety of information that serves as the evidence for determining the illness of the patient P from among a plurality of illness candidates and, if the patient P might be suffering from a plurality of illnesses, enables performing examination for narrowing down the illnesses. Thus, the medical information processing apparatus 20 can effectively utilize the information serving as the evidence in regard to performing diagnosis, and thus support the user to perform diagnosis.

Till now, the explanation was given about identifying a single examination candidate. However, alternatively, the identification function 24d can identify a plurality of examination candidates. For example, as illustrated in FIG. 4B, the identification function 24d identifies three examination candidates, namely, “diagnostic imaging”, “diagnostic imaging+laboratory tests”, and “laboratory tests”. FIG. 4B is a diagram for explaining about the examination candidates according to the first embodiment.

In the case illustrated in FIG. 4B, in the display 22, the output function 24e displays, for example, three examination candidates as identified by the identification function 24d. Then, the user selects one of the three examination candidates. For example, the user selects “diagnostic imaging+laboratory tests” and issues an order for diagnostic imaging and laboratory tests. For example, the user can select the examination candidate by taking into account various aspects such as the physical condition of the patient P, the available manpower, the available rooms, and the available devices. Meanwhile, regarding the orders, the output function 24e can be configured to automatically issue orders.

Moreover, for example, as illustrated in FIG. 4C, the identification function 24d identifies two examination candidates, namely, a “brain tumor analysis application” and a “brain hemorrhage analysis application” based on the scores. For example, the identification function 24d identifies, as the examination candidates, the “brain tumor analysis application” meant for performing detailed examination of “brain tumor” and the “brain hemorrhage analysis application” meant for performing detailed examination of “brain hemorrhage”. FIG. 4C is a diagram for explaining the examination candidates according to the first embodiment. The output function 24e either notifies the user about the fact that the “brain tumor analysis application” and the “brain hemorrhage analysis application” are selected as the examination candidates, or issues orders for an analysis operation.

The output function 24e can issue an order for an analysis operation according to the information collected as the evidence by the scoring function 24c or according to the score-based details. For example, in the case illustrated in FIG. 4C, “age” is collected as part of the information serving as the evidence. In that case, according to the age of the patient P, the output function 24e issues an analysis order after specifying whether to use an adult model or a child model in the “brain tumor analysis application”. Moreover, for example, if the score indicates a possibility of brain infraction, then the output function 24e issues an analysis order after specifying such an application in the “brain tumor analysis application” which is specialized in the analysis of brain infraction.

In the case illustrated in FIG. 4C, regarding the information that serves as the evidence, the medical information processing apparatus 20 not only can use that information for identifying the examination candidates but also can reflect it in the order details. Thus, the medical information processing apparatus 20 can effectively utilize the information serving as the evidence in regard to performing diagnosis, and thus support the user to perform diagnosis.

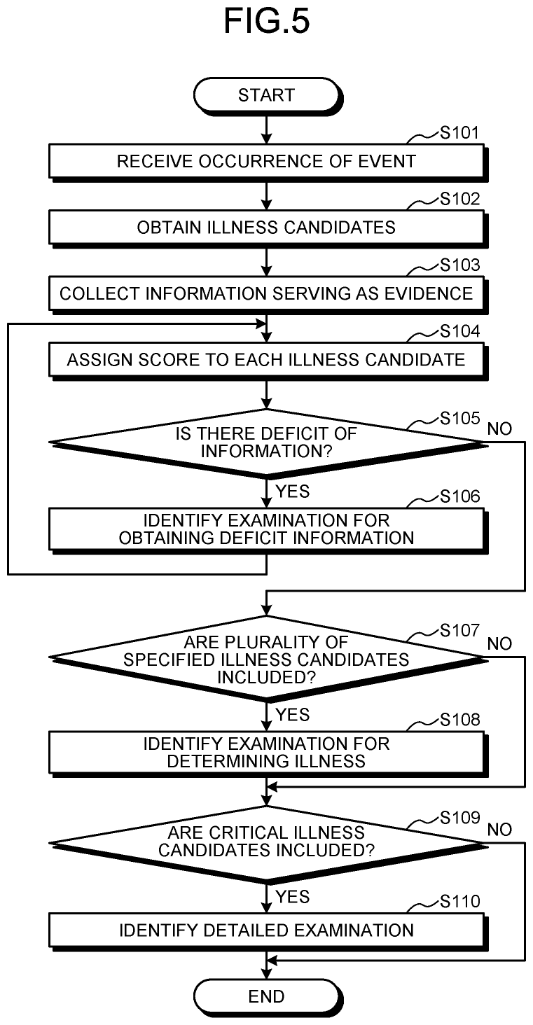

Explained below with reference to FIG. 5 is an exemplary sequence of operations performed in the medical information processing apparatus 20. FIG. 5 is a flowchart for explaining a sequence of operations performed in the medical information processing apparatus 20 according to the first embodiment. The operations at Steps S101 and S102 correspond to the acquisition function 24b. The operations at Steps S103 and S104 correspond to the scoring function 24c. The operations at Steps S105, S106, S107, and S108 correspond to the identification function 24d.

Firstly, the processing circuitry 24 receives the occurrence of an event (Step S101) and obtains a plurality of illness candidates (Step S102). For example, regarding the patient P who has visited the hospital, when either the symptoms or the examination result is registered in a system such as an HIS; the processing circuitry 24 obtains the symptoms or the examination result from the system and obtains a plurality of illness candidates. Moreover, for example, when the user performs an input operation for setting a plurality of illness candidates, the processing circuitry 24 obtains a plurality of illness candidates based on the input operation.

Then, the processing circuitry 24 collects the information serving as the evidence for determining the illness of the patient P (Step S103), and assigns a score to each illness candidate (Step S104). Herein, the processing circuitry 24 determines whether or not there is a deficit of information required to assign the scores (Step S105). If there is a deficit (Yes at Step S105), then the processing circuitry 24 identifies, as the examination candidate, the examination meant for obtaining the deficit information (Step S106).

The processing circuitry 24 performs output based on the examination candidate identified at Step S106. For example, the processing circuitry 24 either notifies the user about the examination candidate or issues an order for examination based on the examination candidate. As a result, the information serving as the evidence gets complemented, and the scoring at Step S104 and the determination at Step S105 is again performed.

If there is no deficit of information (No at Step S105), then the processing circuitry 24 determines whether or not a plurality of specified illness candidates is included (Step S107). That is, the processing circuitry 24 determines whether or not there are two or more illness candidates that, from among a plurality of illness candidates obtained at Step S102, are indicated to be the likely illnesses of the patient P according to the scores. If a plurality of specified illness candidates is included (Yes at Step S107), then the processing circuitry 24 identifies, as the examination candidate, the examination for determining the illness of the patient P from among a plurality of specified illness candidates (Step S108).

Either after the operation at Step S108 is performed or if a plurality of specified illness candidates is not included (No at Step S107), the processing circuitry 24 determines whether or not any critical illness candidates are included (Step S108). That is, the processing circuitry 24 determines whether or not a plurality of illness candidates obtained at Step S102 includes critical illness candidates that are indicated to be the likely illnesses of the patient P according to the scores and that are critical in nature. If critical illness candidates are included (Yes at Step S109), then the processing circuitry 24 identifies, as the examination candidate, the detailed examination of the critical illness candidate (Step S110). Meanwhile, regarding the examination candidates identified at Steps S108 and S110, the processing circuitry 24 can output a new examination candidate as and when identified, or can collectively output all examination candidates after the end of the sequence of operations illustrated in FIG. 5.

As explained above, according to the first embodiment, the acquisition function 24b obtains a plurality of illness candidates. The scoring function 24c collects the information that serves as the evidence for determining the illness of the patient P from among a plurality of illness candidates; and obtains the score for each illness candidate based on the collected information. Then, based on the scores, the identification function 24d identifies the examination candidate meant for supporting the diagnosis of the patient P. The output function 24e performs output based on the examination candidate. As a result, the medical information processing apparatus 20 according to the first embodiment can effectively utilize the information serving as the evidence in regard to performing diagnosis.

Meanwhile, the information serving as the evidence can be collected and utilized by the user too. However, it takes time to manually collect the required volume of information. For example, the user can look into the electronic clinical record for the information about the patient P. However, in the electronic clinical record, the information only about the patient P is mentioned. Hence, in order to refer to the information about the relatives, the user has to take efforts to separately collect the information. Moreover, not only the information serving as the evidence is enormous in volume, but it is also sometimes dispersed across a plurality of systems. Hence, there may be times when some information gets overlooked. In contrast, in the medical information processing apparatus 20, the information serving as the evidence is automatically collected and analyzed, and the output is performed only after the examination candidate is identified. Hence, not only the information serving as the evidence can be utilized in an effective manner, but the volume of information that needs to be handled by the user can also be reduced; so that the burden on the user can be lowered.

Meanwhile, under the circumstances in which definite diagnosis of the illness can be performed using diagnostic imaging; for example, it is also possible to think of a case in which definite diagnosis of the illness can be performed based on the laboratory tests done in the past and the other information serving as the evidence. In that regard, in the medical information processing apparatus 20, since the information serving as the evidence is effectively utilized, unnecessary examination can be avoided.

In the first embodiment, the examination candidate is identified based on the scores obtained by the scoring function 24c, and the output is performed based on the examination candidate. More particularly, in the first embodiment, either the examination candidate is identified and then notified to the user, or an order for examination is issued based on the examination candidate. That is, in the first embodiment, the result of scoring is fed back to the user as the recommended examination candidate or as the examination result. In contrast, in a second embodiment, the explanation is given about a case in which the result of scoring is fed back to a device or an application.

The medical information processing system 1 according to the second embodiment has an identical configuration to the medical information processing system 1 illustrated in FIG. 1. However, the medical information processing system 1 according to the second embodiment need not include the identification function 24d and the output function 24e. In the following explanation, regarding the constituent elements explained in the first embodiment, the same reference numerals are used and the explanation is not repeated.

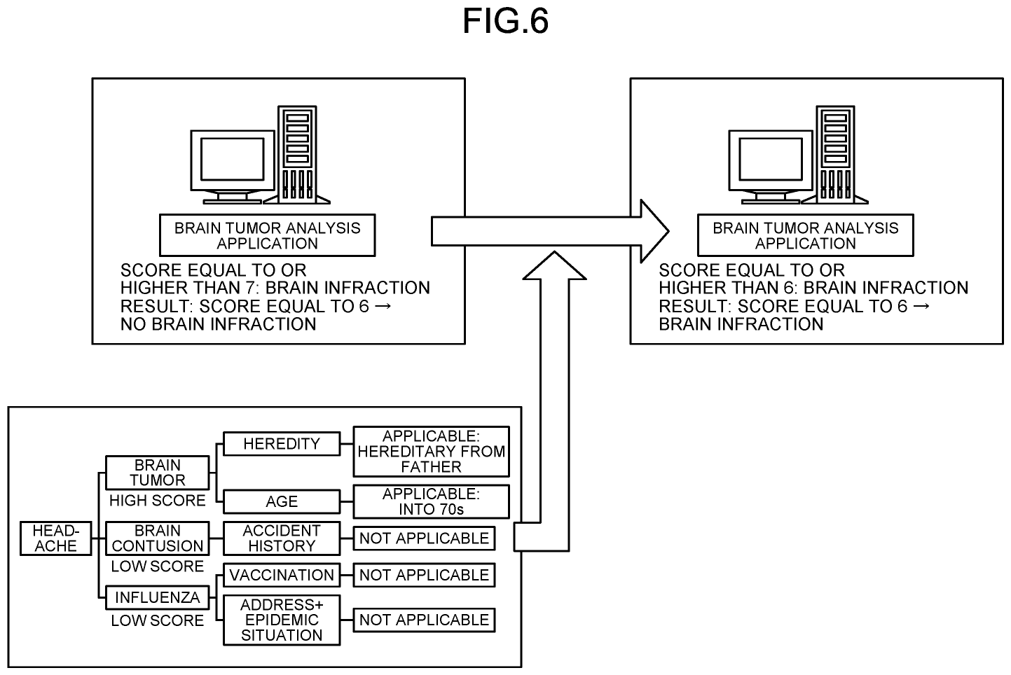

Firstly, the acquisition function 24b obtains a plurality of illness candidates. Then, the scoring function 24c collects the information serving as the evidence for determining the illness of the patient P from among a plurality of illness candidates; and assigns a score to each illness candidate. The following explanation is given for a case in which, as illustrated in FIG. 6, the scores are obtained for three illness candidates, namely, “brain tumor”, “brain contusion”, and “influenza”. FIG. 6 is a diagram illustrating an example of a feedback according to the second embodiment.

The analysis apparatus 40 performs an analysis operation with respect to at least one of a plurality of illness candidates obtained by the acquisition function 24b. For example, as illustrated in FIG. 6, the analysis apparatus 40 executes a brain infraction analysis application with “brain tumor” serving as the target.

An analysis operation such as the brain infraction analysis application includes analysis parameters. For example, in the case of the brain infraction analysis application, medical images of the target region such as the brain are received as input, and a score indicating the state of blood flow is calculated. For example, the brain infraction analysis application receives input of the medical images collected from the patient P, and calculates a score “6” indicating the state of blood flow. In the brain infraction analysis application, a threshold value is set as an analysis parameter; and the score indicating the state of blood flow is compared with the threshold value so as to determine whether or not brain infraction is indicated, and the analysis result is output. For example, as illustrated in the left-side diagram in FIG. 6, in the brain infraction analysis application, a threshold value of “7” is set as an analysis parameter with which the score “6” indicating the state of blood flow is compared; and the analysis result indicating the “no brain infraction” is output.

Herein, based on the score of the illness candidate to be analyzed, the analysis apparatus 40 adjusts the analysis parameters of the analysis operation. For example, in the case illustrated in FIG. 6, the analysis apparatus 40 compares the score “6”, which indicates the state of blood flow based on the medical images collected from the patient P, with the threshold value “7” representing an analysis parameter; and outputs the analysis result indicating “no brain infraction”. On the other hand, the score about “brain tumor” as obtained by the scoring function 24c indicates a high likelihood of brain infraction. In order to resolve such inconsistency, based on the score obtained by the scoring function 24c, the analysis apparatus 40 adjusts the threshold value “7”, which represents an analysis parameter of the brain infraction analysis application, to the value “6”. After the adjustment is done, if the score “6” indicating the state of blood flow is again obtained, then the brain infraction analysis application compares the score with the threshold value “6” and outputs the analysis result indicating “brain infraction” as illustrated in the right-side drawing in FIG. 6.

As explained above, according to the second embodiment, the acquisition function 24b obtains a plurality of illness candidates. The scoring function 24c collects the information serving as the evidence for determining the illness of the patient P from among a plurality of illness candidates; and, based on the collected information, obtains a score for each illness candidate. The analysis apparatus 40 performs an analysis operation with respect to at least one of a plurality of illness candidates. Moreover, based on the score of the illness candidate to be analyzed, the analysis apparatus adjusts the analysis parameters of the analysis operation. As a result, the medical information processing apparatus according to the second embodiment can effectively utilize the information serving as the evidence in regard to performing diagnosis. That is, the medical information processing apparatus 20 can adjust the analysis parameters by utilizing the information serving as the evidence, and thus enhance the accuracy of the analysis operation.

Till now, the explanation was given about the first and second embodiments. Apart from the embodiments described above, various other illustrative embodiments can also be implemented.

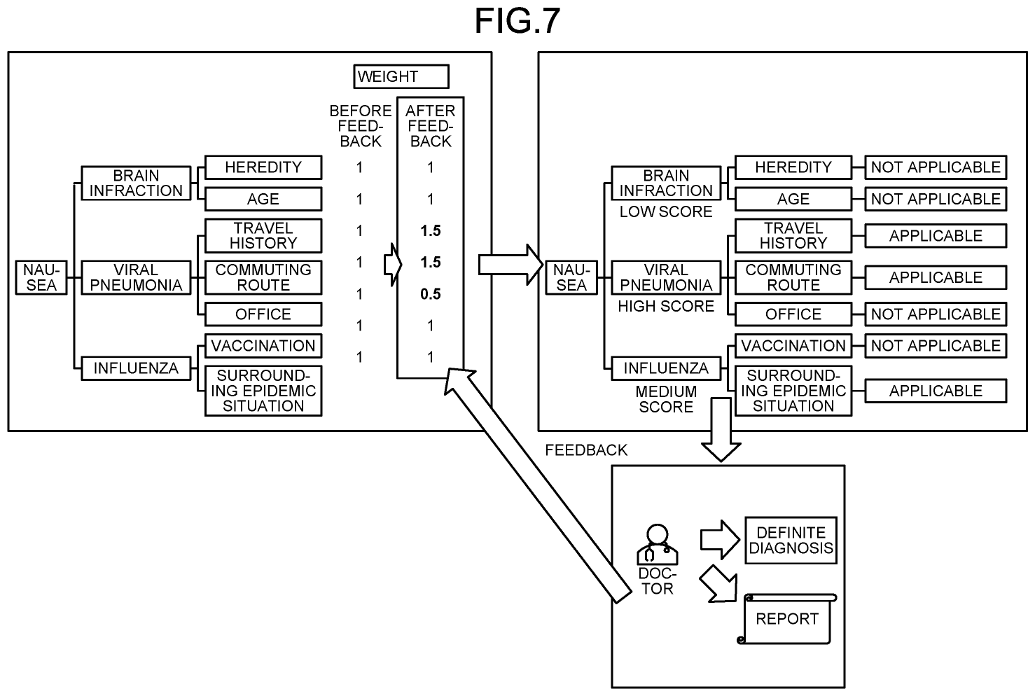

For example, as illustrated in FIG. 7, after the doctor has performed diagnosis, the medical information processing apparatus 20 can reflect the diagnosis result in the score obtaining method. FIG. 7 is a diagram illustrating an example of a feedback according to a third embodiment.

More particularly, based on the chief complaint of “nausea”, the acquisition function 24b obtains a plurality of illness candidates, namely, “brain infraction”, “viral pneumonia”, and “influenza”. Subsequently, the scoring function 24c collects the information such as “heredity”, “age”, “travel history”, “office”, “vaccination”, and “surrounding epidemic situation” as the information serving as the evidence; and obtains the score for each illness candidate. For example, as illustrated in FIG. 7, the scoring function 24c uniformly assigns the weight “1” to each piece of information serving as the evidence, and obtains the score for each illness candidate. Herein, the identification function 24d can identify the examination candidate based on the scores, and the output function 24e can perform output based on the examination candidate. Moreover, based on the score of the illness candidate to be analyzed, the analysis apparatus 40 can adjust the analysis parameters of the analysis operation.

Moreover, as illustrated in FIG. 7, the user who is a doctor performs diagnosis. More particularly, the user performs definite diagnosis and creates a report. Meanwhile, the medical information processing apparatus 20 performs output based on the examination candidate identified according to the scores, and thus can effectively utilize the information serving as the evidence and support the diagnosis of the user. Moreover, the analysis apparatus 40 adjusts the analysis parameters of the analysis operation based on the score, and thus can effectively utilize the information serving as the evidence and enhance the accuracy of the analysis operation. Alternatively, the user can refer to the actual scores obtained by the scoring function 24c and accordingly perform diagnosis.

Subsequently, based on the result of diagnosis of the patient P, the scoring function 24c adjusts the weights exerted on the scores due to each piece of information serving as the evidence. For example, as illustrated in FIG. 7, the scoring function 24c varies the weight assigned to “travel history” from “1” to “1.5”; varies the weight assigned to “commuting route” from “1” to “1.5”; and varies the weight assigned to “office” from “1” to “0.5”. As a result, the scoring function 24c can go on gradually enhancing the scoring accuracy.

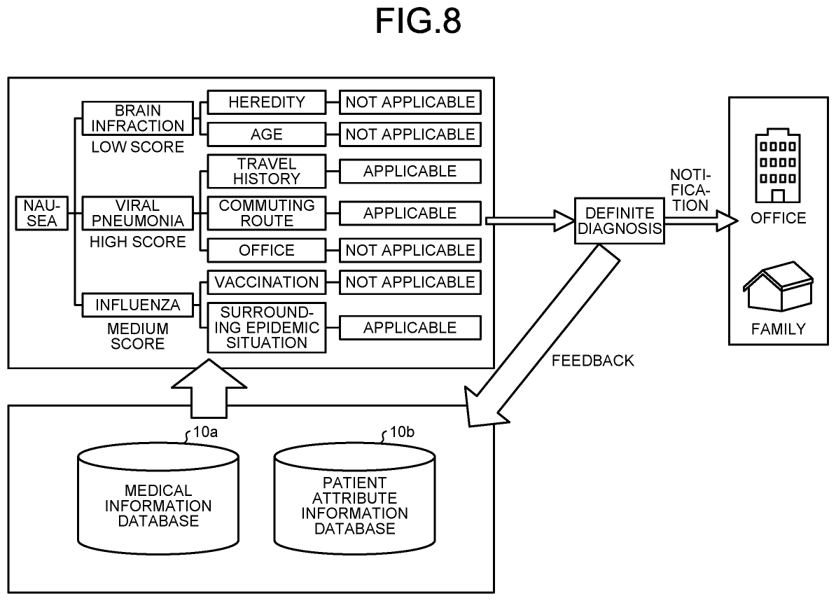

Regarding a feedback of the diagnosis result, another example is explained below with reference to FIG. 8. FIG. 8 is a diagram illustrating an example of a feedback according to the third embodiment. More particularly, the user who is a doctor performs diagnosis in an identical manner to the explanation given with reference to FIG. 7. For example, the user performs definite diagnosis.

The output function 24e notifies the result of diagnosis of the patient P to a related person of the patient P. Herein, a related person implies, for example, an employee of the same office as the patient P, or a family member of the patient P. For example, if the result of diagnosis confirms that the patient P is suffering from an illness of the epidemic nature, then the output function 24e notifies a related person of the patient P. With that, the output function 24e enables prevention of an epidemic of that illness.

Moreover, the output function 24e registers the result of diagnosis of the patient P in a database that is used to manage the information serving as the evidence. For example, based on the result of diagnosis of the patient P, the output function 24e updates the surrounding information of the patient P that is registered in the medical information database 10a, and updates the action information of the patient P that is registered in the patient attribute information database 10b. As a result, the output function 24e can enhance the information serving as the evidence and improve the quality, and in turn can gradually enhance the scoring accuracy.

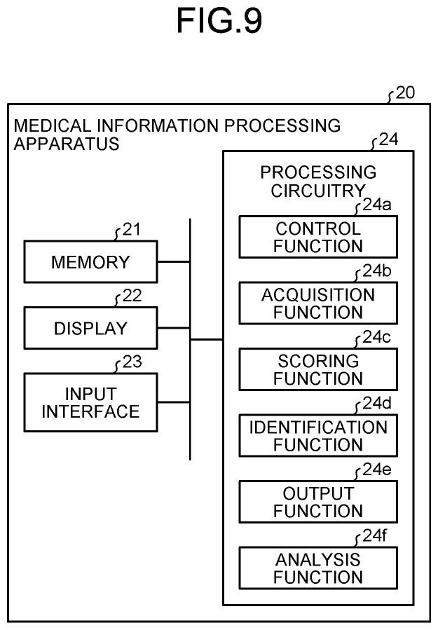

Meanwhile, in the embodiments described above, the analysis apparatus 40 represents an example of the analyzing unit that performs the analysis operation. However, the embodiments are not limited to that example. Alternatively, for example, as illustrated in FIG. 9, the processing circuitry 24 of the medical information processing apparatus 20 can further include an analysis function 24f that is equivalent to the function of the analysis apparatus 40. Thus, the analysis function 24f represents an example of the analyzing unit. FIG. 9 is a block diagram illustrating an exemplary configuration of the medical information processing apparatus 20 according to the third embodiment.

In the explanation given above, the term “processor” implies, for example, a central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)). When the processor is, for example, a CPU; it reads computer programs stored in a memory circuit and executes them so as to implement functions. On the other hand, when the processor is, for example, an ASIC; no computer programs are stored in a memory circuit, but the corresponding functions are directly embedded as logical circuits in the circuit of the processor. Meanwhile, the processors according to the embodiments are not limited to be configured using a single circuit on a processor-by-processor basis. Alternatively, a single processor can be configured by combining a plurality of independent circuits, and the corresponding functions can be implemented. Still alternatively, the constituent elements illustrated in the drawings can be integrated into a single processor, and the corresponding functions can be implemented.

Moreover, with reference to FIG. 1, a single memory 21 is used to store the computer programs corresponding to the processing functions of the processing circuitry 24. However, the embodiments are not limited to that example.

Alternatively, a plurality of memories 21 can be disposed in a dispersed manner, and the processing circuitry 24 can read computer programs from individual memories 21. Still alternatively, instead of storing computer programs in the memory 21, they can be directly incorporated in the circuit of the processor. In that case, the processor reads the computer programs incorporated in its circuit and executes them so as to implement the functions.

The constituent elements of the device illustrated in the drawings are merely conceptual, and need not be physically configured as illustrated. The constituent elements, as a whole or in part, can be separated or integrated either functionally or physically based on various types of loads or use conditions. The processing functions implemented by the device are entirely or partially implemented by the CPU or computer programs that are analyzed and executed by the CPU, or are implemented as hardware by wired logic.

Meanwhile, the medical information processing method explained in the embodiments can be implemented when a medical information processing program, which is written in advance, is executed in a computer such as a personal computer or a workstation. The medical information processing program can be distributed via a network such as the Internet. Alternatively, the medical information program can be recorded in a non-transitory computer-readable recording medium such as a flexible disk (FD), a compact disk read only memory (CD-ROM), a magneto-optical (MO) disk, or a digital versatile disk (DVD). Thus, a computer can read the medical information processing program from a recording medium and execute it.

According to at least one of the embodiments described above, the information serving as the evidence in regard to performing diagnosis can be utilized in an effective manner.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured to
obtain a plurality of illness candidates,
collect information serving as evidence for determining illness of a patient from among the plurality of illness candidates, and obtain a score for each of the plurality of illness candidates based on the information,
identify, based on the scores, an examination candidate meant for supporting diagnosis of the patient, and
perform output based on the examination candidate.

2. The medical information processing apparatus according to claim 1, wherein the processing circuitry notifies a user, who diagnoses the patient, about the examination candidate.

3. The medical information processing apparatus according to claim 1, wherein the processing circuitry issues an order for examination based on the examination candidate.

4. The medical information processing apparatus according to claim 1, further comprising an analysis apparatus that performs an analysis operation with respect to at least one of the plurality of illness candidates, wherein
based on the examination candidate, the processing circuit issues an order to the analysis apparatus for performing the analysis operation.

5. The medical information processing apparatus according to claim 4, wherein, based on details according to either the information or the scores, the processing circuitry issues an order for performing the analysis operation.

6. The medical information processing apparatus according to claim 1, wherein the processing circuitry identifies, as the examination candidate, detailed examination of an illness that, from among the plurality of illness candidates, is an illness candidate indicated to be likely illness of the patient according to the score and that is critical in nature.

7. The medical information processing apparatus according to claim 1, wherein, when there is a plurality of specified illness candidates representing illness candidates indicated to be likely illnesses of the patient according to the scores from among the plurality of illness candidates, the processing circuitry identifies, as the examination candidate, examination for determining illness of the patient from among the plurality of specified illness candidates.

8. The medical information processing apparatus according to claim 1, wherein, when there is a deficit of the information for obtaining the scores, the processing circuitry identifies, as the examination candidate, examination meant for obtaining deficit information.

9. A medical information processing apparatus comprising:
processing circuitry configured to
obtain a plurality of illness candidates, and
collect information serving as evidence for determining illness of a patient from among the plurality of illness candidates, and obtain a score for each of the plurality of illness candidates based on the information; and
an analysis apparatus that performs an analysis operation with respect to at least one of the plurality of illness candidates, wherein
based on the score of illness candidate to be subjected to the analysis operation, the analysis apparatus adjusts analysis parameter of the analysis operation.

10. The medical information processing apparatus according to claim 1, wherein, based on result of diagnosis of the patient, the processing circuitry further adjusts weight exerted on the scores due to each piece of the information.

11. The medical information processing apparatus according to claim 1, wherein the processing circuitry notifies a related person of the patient about result of diagnosis of the patient.

12. The medical information processing apparatus according to claim 1, wherein the processing circuitry registers result of diagnosis of the patient in a database in which the information is managed.

13. The medical information processing apparatus according to claim 1, wherein, based on symptoms of the patient or based on examination result, the processing circuitry obtains the plurality of illness candidates.

14. A medical information processing system comprising processing circuitry configured to
obtain a plurality of illness candidates,
collect information serving as evidence for determining illness of a patient from among the plurality of illness candidates, and obtain a score for each of the plurality of illness candidates based on the information,
identify, based on the scores, an examination candidate meant for supporting diagnosis of the patient, and
perform output based on the examination candidate.

15. A medical information processing system comprising:
processing circuitry configured to
obtain a plurality of illness candidates, and
collect information serving as evidence for determining illness of a patient from among the plurality of illness candidates, and obtain a score for each of the plurality of illness candidates based on the information; and
an analysis apparatus that performs an analysis operation with respect to at least one of the plurality of illness candidates, wherein
based on the score of illness candidate to be subjected to the analysis operation, the analysis apparatus adjusts analysis parameter of the analysis operation.

16. A medical information processing method comprising:
obtaining a plurality of illness candidates;
collecting that includes collecting information serving as evidence for determining illness of a patient from among the plurality of illness candidates, and obtaining a score for each of the plurality of illness candidates based on the information;
identifying, based on the scores, an examination candidate meant for supporting diagnosis of the patient; and
performing output based on the examination candidate.

17. A medical information processing method comprising:
obtaining a plurality of illness candidates;
collecting that includes collecting information serving as evidence for determining illness of a patient from among the plurality of illness candidates, and obtaining a score for each of the plurality of illness candidates based on the information; and
adjusting analysis parameter of an analysis operation, which is performed with respect to at least one of the plurality of illness candidates, based on the score of illness candidate to be subjected to the analysis operation.
